(19) United States
(12) Patent Application Publication
(10) Pub. No.: US 2024/0358455 A1
(43) Pub. Date: Oct. 31, 2024

(54) REDUCED PROFILE ROBOTIC SURGICAL DEVICE AND RELATED SYSTEMS AND METHODS

(71) Applicant: Virtual Incision Corporation, Lincoln, NE (US)

(72) Inventors: Parker Durham, Lincoln, NE (US); Shane Farritor, Lincoln, NE (US); Nathan Wood, Lincoln, NE (US)

(21) Appl. No.: 18/661,197

(22) Filed: May 10, 2024

Related U.S. Application Data
(63) Continuation-in-part of application No. 18/689,586, filed on Jan. 1, 0001.
(60) Provisional application No. 63/384,496, filed on Nov. 21, 2022.

(51) Int. Cl. A61B 34/30; A61B 1/313; A61B 90/00

(52) U.S. Cl. CPC A61B 34/30; A61B 1/3132; A61B 2034/302; A61B 2034/306; A61B 2090/08021

(57) ABSTRACT

Robotic surgical devices having an elongate device body with a distal section having a distal section diameter and a proximal section having a proximal section diameter that is greater than the distal section diameter and first and second arms operably coupled to a distal end of the device body. In some embodiments, the elongate device body has first and second drivetrain assemblies, with both such assemblies having a pitch drivetrain and a roll drivetrain. In other embodiments, the first and second arms each have a forearm having a rotation drivetrain and a roll drivetrain.

### CROSS-REFERENCE TO RELATED APPLICATION(S)

This application is a Continuation-in-Part Application and claims priority to U.S. patent application Ser. No. 18/689,586, filed on Mar. 6, 2024, and entitled “Reduced Profile Robotic Surgical Device and Related Systems and Methods,” which application claims priority to International PCT Application No. PCT/US23/80730, filed on Nov. 21, 2023, which claims the benefit under 35 U.S.C. § 119 (e) to U.S. Provisional Application 63/384,496, filed Nov. 21, 2022 and entitled “Reduced Profile Robotic Surgical Device and Related Systems and Methods,” each of which are hereby incorporated herein by reference in their entireties.

### FIELD

The embodiments disclosed herein relate to various medical devices and related components that can make up a surgical system, including robotic and/or in vivo medical devices and related components. Certain embodiments include various robotic medical devices, including robotic devices that are disposed within a body cavity and positioned using a body or support component disposed through an orifice or opening in the body cavity. Other embodiments relate to various systems that have a robotic surgical device and a controller.

### BACKGROUND

Invasive surgical procedures are essential for addressing various medical conditions. When possible, minimally invasive procedures such as laparoscopy are preferred.

However, known minimally invasive technologies such as laparoscopy are limited in scope and complexity due in part to 1) mobility restrictions resulting from using rigid tools inserted through access ports, and 2) limited visual feedback. Known robotic systems such as the da Vinci® Surgical System (available from Intuitive Surgical, Inc., located in Sunnyvale, CA) are also restricted by the access ports, as well as having the additional disadvantages of being very large, very expensive, unavailable in most hospitals, and having limited sensory and mobility capabilities.

There is a need in the art for improved surgical methods, systems, and devices.

### BRIEF SUMMARY

Discussed herein are various robotic surgical systems having various robotic devices, including reduced-profile robotic surgical devices for use in minimally invasive procedures.

In Example 1, a robotic surgical device comprises an elongate device body, a first arm operably coupled to a distal end of the elongate device body via a first shoulder assembly, and a second arm operably coupled to the distal end of the elongate device body via a second shoulder assembly. The elongate device body comprises a distal section having a distal section width ranging from about 28 mm to about 30 mm and a distal section depth ranging from about 22 mm to about 24 mm and a proximal section having a proximal section diameter that is greater than a distal section diameter.

Example 2 relates to the robotic surgical device according to Example 1, wherein each of the first and second arms has an arm width ranging from about 13 mm to about 15 mm and an arm depth of about 20 mm to about 26 mm.

Example 3 relates to the robotic surgical device according to Example 2, wherein the arm width is about 14 mm and the arm depth is about 23 mm.

Example 4 relates to the robotic surgical device according to Example 1, wherein the distal section width is about 29 mm and the distal section depth is about 23 mm.

Example 5 relates to the robotic surgical device according to Example 1, wherein the proximal section comprises a camera port.

Example 6 relates to the robotic surgical device according to Example 1, wherein the elongate device body further comprises a first drivetrain assembly and a second drivetrain assembly. The first drivetrain assembly comprises a first pitch drivetrain and a first roll drivetrain. The first pitch drivetrain comprises a first pitch actuator, a first pitch motor gear rotatably coupled to the first pitch actuator via a first pitch motor driveshaft, and a first pitch driven gear rotatably coupled to the first pitch motor gear, wherein the first pitch driven gear rotates around an axis that is substantially perpendicular to a longitudinal axis of the first pitch actuator. The first roll drivetrain comprises a first roll actuator disposed proximal of the first pitch actuator, a first roll motor gear rotatably coupled to the first roll actuator, a first roll driveshaft rotatably coupled to the first roll motor gear, wherein the first roll driveshaft is disposed radially adjacent to the first pitch actuator, and a first shoulder driveshaft rotatably coupled to the first roll driveshaft, wherein the first shoulder driveshaft is rotationally constrained to the first shoulder assembly, the first shoulder driveshaft comprising a first lumen defined therethrough, wherein the first pitch motor driveshaft is rotatably disposed through the first lumen, and wherein the first pitch driven gear is rotatably disposed within the first shoulder assembly. The second drivetrain assembly comprises a second pitch drivetrain and a second roll drivetrain. The second pitch drivetrain comprises a second pitch actuator, a second pitch motor gear rotatably coupled to the second pitch actuator via a second pitch motor driveshaft, and a second pitch driven gear rotatably coupled to the second pitch motor gear, wherein the second pitch driven gear rotates around an axis that is substantially perpendicular to a longitudinal axis of the second pitch actuator. The second roll drivetrain comprises a second roll actuator disposed proximal of the second pitch actuator, a second roll motor gear rotatably coupled to the second roll actuator, a second roll driveshaft rotatably coupled to the second roll motor gear, wherein the second roll driveshaft is disposed radially adjacent to the second pitch actuator, and a second shoulder driveshaft rotatably coupled to the second roll driveshaft, wherein the second shoulder driveshaft is rotationally constrained to the second shoulder assembly, the second shoulder driveshaft comprising a second lumen defined therethrough, wherein the second pitch motor driveshaft is rotatably disposed through the second lumen, and wherein the second pitch driven gear is rotatably disposed within the second shoulder assembly.

Example 7 relates to the robotic surgical device according to Example 1, wherein each of the first and second arms comprises an upper arm comprising a rotation drivetrain and a roll drivetrain. The rotation drivetrain comprises a rotation actuator, a rotation motor gear rotatably coupled to the rotation actuator via a rotation motor driveshaft, and a rotation driven gear rotatably coupled to the rotation motor gear, wherein the rotation driven gear rotates around an axis that is substantially perpendicular to a longitudinal axis of the rotation actuator. The roll drivetrain comprises a roll actuator disposed adjacent to the rotation actuator, at least one roll gear rotatably coupled to the roll actuator, and an elbow driveshaft rotatably coupled to the at least one roll gear, wherein the elbow driveshaft is rotationally constrained to the elbow assembly, the elbow driveshaft comprising a lumen defined therethrough, wherein the rotation motor driveshaft is rotatably disposed through the lumen, and wherein the rotation driven gear is rotatably disposed within the elbow assembly.

Example 8 relates to the robotic surgical device according to Example 7, wherein the upper arm further comprises a upper arm housing and a proximal attachment structure disposed at a proximal end of the upper arm housing, wherein the proximal attachment structure is configured to be coupleable to one of the first and second shoulder assemblies, wherein the rotation and roll drivetrains are disposed within the upper arm housing.

Example 9 relates to the robotic surgical device according to Example 7, wherein each of the first and second arms comprises a forearm rotatably coupled to the upper arm and an end effector operably coupled to the forearm.

In Example 10, a robotic surgical device comprises an elongate device body, a first arm, and a second arm. The elongate device body comprises a first drivetrain assembly and a second drivetrain assembly. The first drivetrain assembly comprises a first pitch drivetrain and a first roll drivetrain. The first pitch drivetrain comprises a first pitch actuator, a first pitch motor gear rotatably coupled to the first pitch actuator via a first pitch motor driveshaft; and a first pitch driven gear rotatably coupled to the first pitch motor gear, wherein the first pitch driven gear rotates around an axis that is substantially perpendicular to a longitudinal axis of the first pitch actuator. The first roll drivetrain comprises a first roll actuator disposed proximal of the first pitch actuator, a first roll motor gear rotatably coupled to the first roll actuator, a first roll driveshaft rotatably coupled to the first roll motor gear, wherein the first roll driveshaft is disposed radially adjacent to the first pitch actuator, and a first shoulder driveshaft rotatably coupled to the first roll driveshaft, wherein the first shoulder driveshaft is rotationally constrained to a first shoulder assembly, the first shoulder driveshaft comprising a first lumen defined therethrough, wherein the first pitch motor driveshaft is rotatably disposed through the first lumen, and wherein the first pitch driven gear is rotatably disposed within the first shoulder assembly. The second drivetrain assembly comprise a second pitch drivetrain and a second roll drivetrain. The second pitch drivetrain comprises a second pitch actuator, a second pitch motor gear rotatably coupled to the second pitch actuator via a second pitch motor driveshaft, and a second pitch driven gear rotatably coupled to the second pitch motor gear, wherein the second pitch driven gear rotates around an axis that is substantially perpendicular to a longitudinal axis of the second pitch actuator. The second roll drivetrain comprises a second roll actuator disposed proximal of the second pitch actuator, a second roll motor gear rotatably coupled to the second roll actuator, a second roll driveshaft rotatably coupled to the second roll motor gear, wherein the second roll driveshaft is disposed radially adjacent to the second pitch actuator, a second shoulder driveshaft rotatably coupled to the second roll driveshaft, wherein the second shoulder driveshaft is rotationally constrained to a second shoulder assembly, the second shoulder driveshaft comprising a second lumen defined therethrough, wherein the second pitch motor driveshaft is rotatably disposed through the second lumen, and wherein the second pitch driven gear is rotatably disposed within the second shoulder assembly. The first arm is operably coupled to the first shoulder assembly, and the second arm is operably coupled to the second shoulder assembly.

Example 11 relates to the robotic surgical device according to Example 10, wherein the elongate device body further comprises a distal section having a distal section width ranging from about 28 mm to about 30 mm and a distal section depth ranging from about 22 mm to about 24 mm, and a proximal section having a proximal section diameter that is greater than a distal section diameter.

Example 12 relates to the robotic surgical device according to Example 11, wherein the distal section width is about 29 mm and the distal section depth is about 23 mm.

Example 13 relates to the robotic surgical device according to Example 11, wherein the proximal section comprises a camera port.

Example 14 relates to the robotic surgical device according to Example 10, wherein each of the first and second arms has an arm width ranging from about 13 mm to about 15 mm and an arm depth of about 20 mm to about 26 mm.

Example 15 relates to the robotic surgical device according to Example 14, wherein the arm width is about 14 mm and the arm depth is about 23 mm.

Example 16 relates to the robotic surgical device according to Example 10, wherein each of the first and second arms comprises an upper arm comprising a rotation drivetrain and a roll drivetrain. The rotation drivetrain comprises a rotation actuator, a rotation motor gear rotatably coupled to the rotation actuator via a rotation motor driveshaft, and a rotation driven gear rotatably coupled to the rotation motor gear, wherein the rotation driven gear rotates around an axis that is substantially perpendicular to a longitudinal axis of the rotation actuator. The roll drivetrain comprises a roll actuator disposed adjacent to the rotation actuator, at least one roll gear rotatably coupled to the roll actuator, and an elbow driveshaft rotatably coupled to the at least one roll gear, wherein the elbow driveshaft is rotationally constrained to the elbow assembly, the elbow driveshaft comprising a lumen defined therethrough, wherein the rotation motor driveshaft is rotatably disposed through the lumen, and wherein the rotation driven gear is rotatably disposed within the elbow assembly.

Example 17 relates to the robotic surgical device according to Example 16, wherein the upper arm further comprises a upper arm housing and a proximal attachment structure disposed at a proximal end of the upper arm housing, wherein the proximal attachment structure is configured to be coupleable to one of the first and second shoulder assemblies, wherein the rotation and roll drivetrains are disposed within the upper arm housing.

Example 18 relates to the robotic surgical device according to Example 16, wherein each of the first and second arms comprises a forearm rotatably coupled to the upper arm and an end effector operably coupled to the forearm.

In Example 19, a robotic surgical device comprises an elongate device body, a first arm operably coupled to a first shoulder assembly, and a second arm operably coupled to the second shoulder assembly. The elongate device body comprises a device body housing, a first drivetrain assembly disposed within the device body housing, and a second drivetrain assembly disposed within the device body housing. The device body housing comprises a distal section having a distal section diameter and a proximal section having a proximal section diameter that is greater than the distal section diameter. The first drivetrain assembly comprises a first pitch drivetrain and a first roll drivetrain. The first pitch drivetrain comprises a first pitch actuator, a first pitch motor gear rotatably coupled to the first pitch actuator via a first pitch motor driveshaft, and a first pitch driven gear rotatably coupled to the first pitch motor gear, wherein the first pitch driven gear rotates around an axis that is substantially perpendicular to a longitudinal axis of the first pitch actuator. The first roll drivetrain comprises a first roll actuator disposed proximal of the first pitch actuator, a first roll motor gear rotatably coupled to the first roll actuator, a first roll driveshaft rotatably coupled to the first roll motor gear, wherein the first roll driveshaft is disposed radially adjacent to the first pitch actuator, and a first shoulder driveshaft rotatably coupled to the first roll driveshaft, wherein the first shoulder driveshaft is rotationally constrained to a first shoulder assembly, the first shoulder driveshaft comprising a first lumen defined therethrough, wherein the first pitch motor driveshaft is rotatably disposed through the first lumen, and wherein the first pitch driven gear is rotatably disposed within the first shoulder assembly. The second drivetrain assembly is disposed within the device body housing and comprises a second pitch drivetrain and a second roll drivetrain. The second pitch drivetrain comprises a second pitch actuator, a second pitch motor gear rotatably coupled to the second pitch actuator via a second pitch motor driveshaft, and a second pitch driven gear rotatably coupled to the second pitch motor gear, wherein the second pitch driven gear rotates around an axis that is substantially perpendicular to a longitudinal axis of the second pitch actuator. The second roll drivetrain comprises a second roll actuator disposed proximal of the second pitch actuator, a second roll motor gear rotatably coupled to the second roll actuator, a second roll driveshaft rotatably coupled to the second roll motor gear, wherein the second roll driveshaft is disposed radially adjacent to the second pitch actuator, and a second shoulder driveshaft rotatably coupled to the second roll driveshaft, wherein the second shoulder driveshaft is rotationally constrained to a second shoulder assembly, the second shoulder driveshaft comprising a second lumen defined therethrough, wherein the second pitch motor driveshaft is rotatably disposed through the second lumen, and wherein the second pitch driven gear is rotatably disposed within the second shoulder assembly.

While multiple embodiments are disclosed, still other embodiments will become apparent to those skilled in the art from the following detailed description, which shows and describes illustrative embodiments. As will be realized, the various embodiments are capable of modifications in various obvious aspects, all without departing from the spirit and scope thereof. Accordingly, the drawings and detailed description are to be regarded as illustrative in nature and not restrictive.

### BRIEF DESCRIPTION OF THE DRAWINGS

FIG. 1 is a perspective view of a robotic surgical system.

FIG. 2 is a perspective view of a robotic surgical device.

FIG. 3A is a front view of a robotic surgical device, according to one embodiment.

FIG. 3B is a side view of the robotic surgical device of FIG. 3A, according to one embodiment.

FIG. 4 is a front view of the arms of a robotic surgical device, according to one embodiment.

FIG. 5 is a side view of the workspace of a robotic surgical device, according to one embodiment.

FIG. 6A is a perspective view of the workspace of a robotic surgical device in which the arms are disposed at the forward sweep angle, according to one embodiment.

FIG. 6B is a perspective view of the workspace of the robotic surgical device of FIG. 6A in which the arms are disposed at the neutral position, according to one embodiment.

FIG. 6C is a perspective view of the workspace of the robotic surgical device of FIG. 6A in which the arms are disposed at the rearward sweep angle, according to one embodiment.

FIG. 7 is a perspective view of the housing of an elongate body of a robotic surgical device, according to one embodiment.

FIG. 8A is a perspective view of some of the internal components of the housing of an elongate body of a robotic surgical device, according to one embodiment.

FIG. 8B is a perspective view of the right and left drivetrains of the elongate body of FIG. 8A, according to one embodiment.

FIG. 8C is a perspective view of the right shoulder roll drivetrain of the elongate body of FIG. 8A, according to one embodiment.

FIG. 8D is a perspective view of the right shoulder pitch drivetrain of the elongate body of FIG. 8A, according to one embodiment.

FIG. 9A is a perspective view of a right upper arm, according to one embodiment.

FIG. 9B is a perspective view of the roll and rotation drivetrains of the right upper arm of FIG. 9A, according to one embodiment.

FIG. 9C is a perspective view of the roll drivetrain of the right upper arm of FIG. 9A, according to one embodiment.

FIG. 9D is a perspective view of the rotation drivetrain of the right upper arm of FIG. 9A, according to one embodiment.

FIG. 10A is a perspective view of a right forearm, according to one embodiment.

FIG. 10B is a perspective view of some of the internal components of the right forearm of FIG. 10A, according to one embodiment.

FIG. 10C is a perspective view of the right forearm of FIG. 10A with an end effector attached thereto, according to one embodiment.

FIG. 10D is a side view of the internal components of the right forearm of FIG. 10A, according to one embodiment.

FIG. 10E is an opposing side view of the internal components of the right forearm of FIG. 10A, according to one embodiment.

FIG. 10F is an end view of the internal components of the right forearm of FIG. 10A, according to one embodiment.

FIG. 10G is a perspective view of a cross-sectional view of the distal end of the right forearm of FIG. 10A with an end effector disposed for insertion therein, according to one embodiment.

FIG. 10H is a perspective view of some of the internal components of the right forearm of FIG. 10A with an end effector attached thereto, according to one embodiment.

FIG. 10I is a perspective view of the internal components of FIG. 10H, according to one embodiment.

FIG. 10J is a perspective view of other internal components of the right forearm of FIG. 10A with an end effector attached thereto, according to one embodiment.

FIG. 10K is a perspective view of the internal components of FIG. 10J, according to one embodiment.

FIG. 10L is a perspective view some of the electrical components of the right forearm of FIG. 10A with an end effector attached thereto, according to one embodiment.

FIG. 11A is a cross-sectional schematic depiction of a right upper arm with motors disposed therein, according to one embodiment.

FIG. 11B is a cross-section schematic depiction of the right upper arm, according to one embodiment.

FIG. 11C is another cross-section schematic depiction of the right upper arm, according to one embodiment.

FIG. 12A is a front view of a robotic device with the arms in an active configuration, according to one embodiment.

FIG. 12B is a front view of a robotic device with the arms in an insertion configuration, according to one embodiment.

FIG. 12C is a side view of a robotic device with the arms in an insertion configuration, according to one embodiment.

FIG. 12D is an end view of a robotic device with the arms in an insertion configuration, according to one embodiment.

FIG. 12E is a cross sectional view of the arms and narrow body portion of a robotic device with the arms in an insertion configuration, according to one embodiment.

FIG. 12F is a cross sectional view of the internal components of the arms of a robotic device with the arms in an insertion configuration, according to one embodiment.

FIG. 13 is a perspective view of a one-arm robotic device, according to one embodiment.

FIG. 14A is a perspective view of a three-arm robotic device with the arms in a bent configuration, according to one embodiment.

FIG. 14B is a perspective view of the three-arm robotic device of FIG. 13A with the arms in a straight configuration, according to one embodiment.

### DETAILED DESCRIPTION

The various systems and devices disclosed herein relate to devices for use in medical procedures and systems. More specifically, various embodiments relate to various medical devices, including robotic devices and related methods and systems.

It is understood that the various embodiments of robotic devices and related methods and systems disclosed herein can be incorporated into or used with any other known medical devices, systems, and methods. For example, the various embodiments disclosed herein may be incorporated into or used with any of the medical devices and systems disclosed in U.S. Pat. No. 8,968,332 (issued on Mar. 3, 2015 and entitled “Magnetically Coupleable Robotic Devices and Related Methods”), U.S. Pat. No. 8,834,488 (issued on Sep. 16, 2014 and entitled “Magnetically Coupleable Surgical Robotic Devices and Related Methods”), U.S. Pat. No. 10,307,199 (issued on Jun. 4, 2019 and entitled “Robotic Surgical Devices and Related Methods”), U.S. Pat. No. 9,579,088 (issued on Feb. 28, 2017 and entitled “Methods, Systems, and Devices for Surgical Visualization and Device Manipulation”), U.S. Patent Application 61/030,588 (filed on Feb. 22, 2008), U.S. Pat. No. 8,343,171 (issued on Jan. 1, 2013 and entitled “Methods and Systems of Actuation in Robotic Devices”), U.S. Pat. No. 8,828,024 (issued on Sep. 9, 2014 and entitled “Methods and Systems of Actuation in Robotic Devices”), U.S. Pat. No. 9,956,043 (issued on May 1, 2018 and entitled “Methods and Systems of Actuation in Robotic Devices”), U.S. patent application Ser. No. 15/966,606 (filed on Apr. 30, 2018 and entitled “Methods, Systems, and Devices for Surgical Access and Procedures”), U.S. patent application Ser. No. 12/192,663 (filed on Aug. 15, 2008 and entitled “Medical Inflation, Attachment, and Delivery Devices and Related Methods”), U.S. patent application Ser. No. 15/018,530 (filed on Feb. 8, 2016 and entitled “Medical Inflation, Attachment, and Delivery Devices and Related Methods”), U.S. Pat. No. 8,974,440 (issued on Mar. 10, 2015 and entitled “Modular and Cooperative Medical Devices and Related Systems and Methods”), U.S. Pat. No. 8,679,096 (issued on Mar. 25, 2014 and entitled “Multifunctional Operational Component for Robotic Devices”), U.S. Pat. No. 9,179,981 (issued on Nov. 10, 2015 and entitled “Multifunctional Operational Component for Robotic Devices”), U.S. Pat. No. 9,883,911 (issued on Feb. 6, 2018 and entitled “Multifunctional Operational Component for Robotic Devices”), U.S. patent application Ser. No. 15/888,723 (filed on Feb. 5, 2018 and entitled “Multifunctional Operational Component for Robotic Devices”), U.S. Pat. No. 8,894,633 (issued on Nov. 25, 2014 and entitled “Modular and Cooperative Medical Devices and Related Systems and Methods”), U.S. Pat. No. 8,968,267 (issued on Mar. 3, 2015 and entitled “Methods and Systems for Handling or Delivering Materials for Natural Orifice Surgery”), U.S. Pat. No. 9,060,781 (issued on Jun. 23, 2015 and entitled “Methods, Systems, and Devices Relating to Surgical End Effectors”), U.S. Pat. No. 9,757,187 (issued on Sep. 12, 2017 and entitled “Methods, Systems, and Devices Relating to Surgical End Effectors”), U.S. Pat. No. 10,350,000 (issued on Jul. 16, 2019 and entitled “Methods, systems, and devices relating to surgical end effectors”), U.S. patent application Ser. No. 16/512,510 (filed on Jul. 16, 2019 and entitled “Methods, Systems, and Devices Relating to Surgical End Effectors”), U.S. Pat. No. 9,089,353 (issued on Jul. 28, 2015 and entitled “Robotic Surgical Devices, Systems, and Related Methods”), U.S. Pat. No. 10,111,711 (issued on Oct. 30, 2018 and entitled “Robotic Surgical Devices, Systems, and Related Methods”), U.S. patent application Ser. No. 16/123,619 (filed on Sep. 6, 2018 and entitled “Robotic Surgical Devices, Systems and Related Methods”), U.S. Pat. No. 9,770,305 (issued on Sep. 26, 2017 and entitled “Robotic Surgical Devices, Systems, and Related Methods”), U.S. patent application Ser. No. 15/661,147 (filed on Jul. 27, 2017 and entitled “Robotic Devices with On Board Control & Related Systems & Devices”), U.S. patent application Ser. No. 13/833,605 (filed on Mar. 15, 2013 and entitled “Robotic Surgical Devices, Systems, and Related Methods”), U.S. patent application Ser. No. 13/738,706 (filed on Jan. 10, 2013 and entitled “Methods, Systems, and Devices for Surgical Access and Insertion”), U.S. patent application Ser. No. 14/661,465 (filed on Mar. 18, 2015 and entitled “Methods, Systems, and Devices for Surgical Access and Insertion”), U.S. patent application Ser. No. 15/890,860 (filed on Feb. 7, 2018 and entitled “Methods, Systems, and Devices for Surgical Access and Insertion”), U.S. Pat. No. 9,498,292 (issued on Nov. 22, 2016 and entitled “Single Site Robotic Devices and Related Systems and Methods”), U.S. Pat. No. 10,219,870 (issued on Mar. 5, 2019 and entitled “Single site robotic device and related systems and methods”), U.S. patent application Ser. No. 16/293,135 (filed Mar. 3, 2019 and entitled “Single Site Robotic Device and Related Systems and Methods”), U.S. Pat. No. 9,010,214 (issued on Apr. 21, 2015 and entitled “Local Control Robotic Surgical Devices and Related Methods”), U.S. Pat. No. 10,470,828 (issued on Nov. 12, 2019 and entitled “Local Control Robotic Surgical Devices and Related Methods”), U.S. patent application Ser. No. 16/596,034 (filed on Oct. 8, 2019 and entitled “Local Control Robotic Surgical Devices and Related Methods”), U.S. Pat. No. 9,743,987 (issued on Aug. 29, 2017 and entitled “Methods, Systems, and Devices Relating to Robotic Surgical Devices, End Effectors, and Controllers”), U.S. patent application Ser. No. 15/687,787 (filed on Aug. 28, 2017 and entitled “Methods, Systems, and Devices Relating to Robotic Surgical Devices, End Effectors, and Controllers”), U.S. Pat. No. 9,888,966 (issued on Feb. 13, 2018 and entitled “Methods, Systems, and Devices Relating to Force Control Surgical Systems”), U.S. patent application Ser. No. 15/894,489 (filed on Feb. 12, 2018 and entitled “Methods, Systems, and Devices Relating to Force Control Surgical Systems”), U.S. patent application Ser. No. 14/212,686 (filed on Mar. 14, 2014 and entitled “Robotic Surgical Devices, Systems, and Related Methods”), U.S. patent application Ser. No. 14/334,383 (filed on Jul. 17, 2014 and entitled “Robotic Surgical Devices, Systems, and Related Methods”), U.S. patent application Ser. No. 14/853,477 (filed on Sep. 14, 2015 and entitled “Quick-Release End Effectors and Related Systems and Methods”), U.S. patent application Ser. No. 16/504,793 (filed on Jul. 8, 2019 and entitled “Quick-Release End Effectors and Related Systems and Methods”), U.S. Pat. No. 10,376,322 (issued on Aug. 13, 2019 and entitled “Robotic Device with Compact Joint Design and Related Systems and Methods”), U.S. patent application Ser. No. 16/538,902 (filed on Aug. 13, 2019 and entitled “Robotic Device with Compact Joint Design and Related Systems and Methods”), U.S. patent application Ser. No. 15/227,813 (filed on Aug. 3, 2016 and entitled Robotic Surgical Devices, System and Related Methods”) U.S. patent application Ser. No. 15/599,231 (filed on May 18, 2017 and entitled “Robotic Surgical Devices, Systems, and Related Methods”), U.S. patent application Ser. No. 15/687,113 (filed on Aug. 25, 2017 and entitled “Quick-Release End Effector Tool Interface”), U.S. patent application Ser. No. 15/691,087 (filed on Aug. 30, 2017 and entitled “Robotic Device with Compact Joint Design and an Additional Degree of Freedom and Related Systems and Methods”), U.S. patent application Ser. No. 15/821,169 (filed on Nov. 22, 2017 and entitled “Gross Positioning Device and Related Systems and Methods”), U.S. patent application Ser. No. 15/826,166 (filed on Nov. 29, 2017 and entitled “User controller with user presence detection and related systems and methods”), U.S. patent application Ser. No. 15/842,230 (filed on Dec. 14, 2017 and entitled “Releasable Attachment Device for Coupling to Medical Devices and Related Systems and Methods”), U.S. patent application Ser. No. 16/144,807 (filed on Sep. 27, 2018 and entitled “Robotic Surgical Devices with Tracking Camera Technology and Related Systems and Methods”), U.S. patent application Ser. No. 16/241,263 (filed on Jan. 7, 2019 and entitled “Single-Manipulator Robotic Device With Compact Joint Design and Related Systems and Methods”), U.S. patent application Ser. No. 16/736,329 (filed on Jan. 7, 2020 and entitled “Robotically Assisted Surgical System and Related Devices and Methods”), U.S. patent application Ser. No. 17/368,255 (filed on Jul. 6, 2021 and entitled “Surgical Robot Positioning System and Related Devices and Methods”), U.S. Pat. No. 18/064,784 (filed on Dec. 12, 2022 and entitled “Robotic Arm with Hybrid Actuation Assemblies and Related Devices, Systems, and Methods”), U.S. patent application Ser. No. 18/299,129 (filed on Apr. 12, 2023 and entitled “Energy Coupling Mitigation Device and Related Systems and Methods”), U.S. Provisional Application 63/384,464, filed on Nov. 21, 2022, U.S. Provisional Application 63/384,502, filed on Nov. 21, 2022, U.S. Pat. No. 7,492,116 (filed on Oct. 31, 2007 and entitled “Robot for Surgical Applications”), U.S. Pat. No. 7,772,796 (filed on Apr. 3, 2007 and entitled “Robot for Surgical Applications”), and U.S. Pat. No. 8,179,073 (issued on May 15, 2011, and entitled “Robotic Devices with Agent Delivery Components and Related Methods”), all of which are hereby incorporated herein by reference in their entireties.

Certain device and system implementations disclosed in the applications listed above can be positioned within a body cavity of a patient, or a portion of the device can be placed within the body cavity, in combination with a support component similar to those disclosed herein. An “in vivo device” as used herein means any device that can be positioned, operated, or controlled at least in part by a user while being positioned within a body cavity of a patient, including any device that is coupled to a support component such as a rod or other such component that is disposed through an opening or orifice of the body cavity, also including any device positioned substantially against or adjacent to a wall of a body cavity of a patient, further including any such device that is internally actuated (having no external source of motive force), and additionally including any device that may be used laparoscopically or endoscopically during a surgical procedure. As used herein, the terms “robot,” and “robotic device” shall refer to any device that can perform a task either automatically or in response to a command.

Certain embodiments provide for insertion of the present invention into the cavity while maintaining sufficient insufflation of the cavity. Further embodiments minimize the physical contact of the surgeon or surgical users with the present invention during the insertion process. Other implementations enhance the safety of the insertion process for the patient and the present invention. For example, some embodiments provide visualization of the present invention as it is being inserted into the patient's cavity to ensure that no damaging contact occurs between the system/device and the patient. In addition, certain embodiments allow for minimization of the incision size/length. Other implementations include devices that can be inserted into the body via an incision or a natural orifice. Further implementations reduce the complexity of the access/insertion procedure and/or the steps required for the procedure. Other embodiments relate to devices that have minimal profiles, minimal size, or are generally minimal in function and appearance to enhance ease of handling and use.

As in manual laparoscopic procedures, a known insufflation system can be used to pump sterile carbon dioxide (or other gas) into the patient's abdominal cavity. This lifts the abdominal wall from the organs and creates space for the robot. In certain implementations, the system has no direct interface with the insufflation system. Alternatively, the system can have a direct interface to the insufflation system.

In certain implementations in which the device is inserted through an insertion port, the insertion port is a known, commercially-available flexible membrane placed transabdominally to seal and protect the abdominal incision. This off-the-shelf component is the same device or substantially the same device that is used in substantially the same way for Hand-Assisted Laparoscopic Surgery (HALS). The only difference is that the arms of the robotic device according to the various embodiments herein are inserted into the abdominal cavity through the insertion port rather than the surgeon's hand. The robotic device body seals against the insertion port when it is positioned therethrough, thereby maintaining insufflation pressure. The port is single-use and disposable. Alternatively, any known port can be used. In further alternatives, the device can be inserted through an incision without a port or through a natural orifice.

Certain implementations disclosed herein relate to “combination” or “modular” medical devices that can be assembled in a variety of configurations. For purposes of this application, both “combination device” and “modular device” shall mean any medical device having modular or interchangeable components that can be arranged in a variety of different configurations.

Certain embodiments disclosed or contemplated herein can be used for colon resection, a surgical procedure performed to treat patients with lower gastrointestinal diseases such as diverticulitis, Crohn's disease, inflammatory bowel disease and colon cancer. Approximately two-thirds of known colon resection procedures are performed via a completely open surgical procedure involving an 8-to 12-inch incision and up to six weeks of recovery time. Because of the complicated nature of the procedure, existing robot-assisted surgical devices are rarely used for colon resection surgeries, and manual laparoscopic approaches are only used in one-third of cases. In contrast, the various implementations disclosed herein can be used in a minimally invasive approach to a variety of procedures that are typically performed ‘open’ by known technologies, with the potential to improve clinical outcomes and health care costs. Further, the various implementations disclosed herein can be used for any laparoscopic surgical procedure in place of the known mainframe-like laparoscopic surgical robots that reach into the body from outside the patient. That is, the less-invasive robotic systems, methods, and devices disclosed herein feature small, self-contained surgical devices that are inserted in their entireties through a single incision in the patient's abdomen. Designed to utilize existing tools and techniques familiar to surgeons, the devices disclosed herein will not require a dedicated operating room or specialized infrastructure, and, because of their much smaller size, are expected to be significantly less expensive than existing robotic alternatives for laparoscopic surgery. Due to these technological advances, the various embodiments herein could enable a minimally invasive approach to procedures performed in open surgery today.

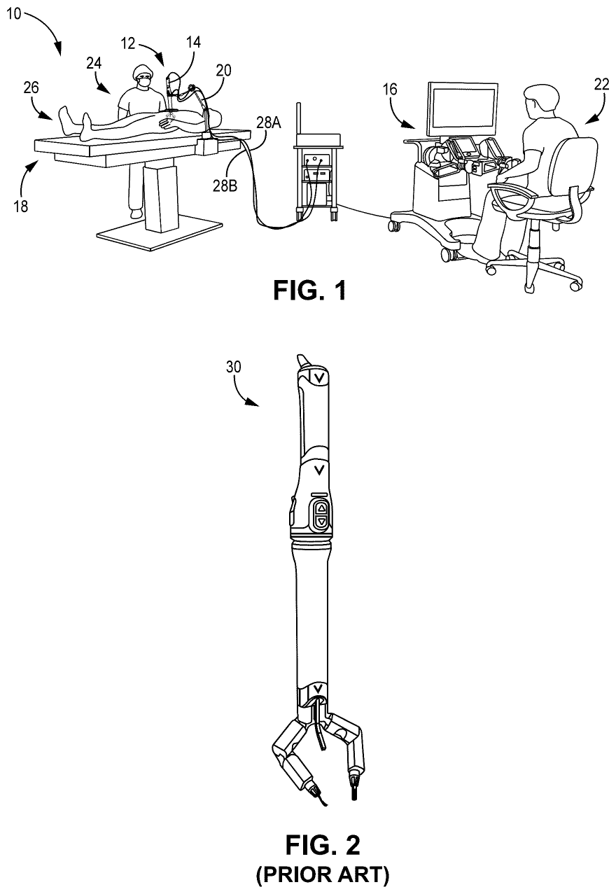

FIG. 1 depicts one embodiment of a robotic surgical system 10 having several components that will be described in additional detail below. The components of the various system implementations disclosed or contemplated herein can include an external control console 16 and a robotic device 12 having a removable camera 14 as will also be described in additional detail below. In accordance with the implementation of FIG. 1, the robotic device 12 is shown mounted to the operating table 18 via a known, commercially available support arm 20 and positioned such that a portion of the device 12 is disposed within a cavity of a patient 26 via an incision. The system 10 can be, in certain implementations, operated by the surgeon 22 at the console 16 and one surgical assistant 24 positioned at the operating table 18. Alternatively, one surgeon 22 can operate the entire system 10. In a further alternative, three or more people can be involved in the operation of the system 10. It is further understood that the surgeon (or user) 22 can be located at a remote location in relation to the operating table 18 such that the surgeon 22 can be in a different city or country or on a different continent from the patient on the operating table 18.

In this specific implementation, the robotic device 12 with the camera 14 are both connected to the surgeon console 16 via cables: a device cable 28A and a camera cable 28B that will be described in additional detail below. Alternatively, any connection configuration can be used. In certain implementations, the system can also interact with other devices during use such as a electrosurgical generator, an insertion port, and auxiliary monitors.

One known robotic device 30 that can be used in the system 10 described above is depicted in FIG. 2. The device 30 has two robotic arms with end effectors coupled thereto that can be used to perform various procedures within a target cavity of a patient (such as the patient 26 in FIG. 1).

The various device embodiments disclosed or contemplated herein have several differences in comparison to the known device 30, including drivetrain configurations, robotic arm configurations, and overall dimensions (such as, for example, a device body with a smaller radial diameter, as will be discussed in further detail below).

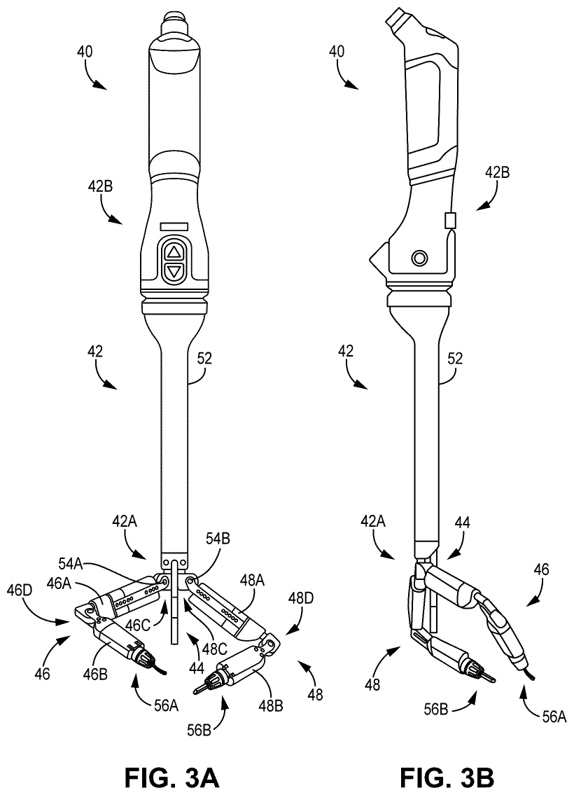

FIGS. 3A and 3B depict one exemplary implementation of an improved robotic device 40 that can be incorporated into the exemplary system 10 discussed above or any other system disclosed or contemplated herein. The device 40 has a body (or “torso”) 42 having a distal end 42A and proximal end 42B, with the imaging device (or “camera”) 44 disposed therethrough, as mentioned above and as will be described in additional detail below. Briefly, the robotic device 40 has two robotic arms 46, 48 operably coupled thereto and the camera 44 is removably positionable through the body 42 and disposed between the two arms 46, 48. That is, device 40 has a first (or “right”) arm 46 and a second (or “left) arm 48, both of which are operably coupled to the device 40 as discussed in additional detail below. In this embodiment, the body 42 of the device 40 as shown has an enclosure (also referred to as a “cover” or “casing”) 52 such that the internal components and lumens of the body 42 are disposed within the enclosure 52. The device body 42 has two rotatable bodies (also referred to as “shoulders” or “turrets”) 54A, 54B: a first (or “right”) shoulder 54A and a second (or “left”) shoulder 54B. Each arm 46, 48 in this implementation also has an upper arm (also referred to herein as an “inner arm,” “inner arm assembly,” “inner link,” “inner link assembly,” “upper arm assembly,” “first link,” or “first link assembly”) 46A, 48A, and a forearm (also referred to herein as an “outer arm,” “outer arm assembly,” “outer link,” “outer link assembly,” “forearm assembly,” “second link,” or “second link assembly”) 46B, 48B. The right upper arm 46A is operably coupled to the right shoulder 54A of the body 42 at the right shoulder joint 46C and the left upper arm 48A is operably coupled to the left shoulder 54B of the body 42 at the left shoulder joint 48C. Further, for each arm 46, 48, the forearm 46B, 48B is rotatably coupled to the upper arm 46A, 48A at the elbow joint 46D, 48D. In various embodiments, the forearms 46B, 48B are configured to receive various removeable, interchangeable end effectors 56A, 56B.

The end effectors 56A, 56B on the distal end of the arms 46, 48 can be various tools 56A, 56B (scissors, graspers, needle drivers and the like), as will be described in additional detail below. In certain implementations, the tools 56A, 56B are designed to be removable, including in some instances by a small twist of the tool knob that couples the end effector 56A, 56B to the arm 46, 48. In certain implementations, at least two single-use, interchangeable, disposable surgical end effectors can be used with any of the robotic device embodiments herein (including device 40). Such end effectors can include, but are not limited to, a fenestrated grasper capable of bi-polar cautery, scissors that deliver mono-polar cautery, a hook that delivers mono-polar cautery, and a left/right needle driver set. The tools can be selected for the specific surgical task. Certain forearm and end effector configurations that allow for the removability and interchangeability of the end effectors are disclosed in detail in U.S. application Ser. No. 14/853,477, which is incorporated by reference above. Further, it is understood that any known forearm and end effector combinations can be used in any of the robotic device embodiments disclosed or contemplated herein.

In various implementations, at least one of the body 42 and each of the links of the arms 46, 48 can contain a variety of actuators or motors. In certain implementations, the body 42 has no motors disposed therein, while there is at least one motor in each of the arms 46, 48. Alternatively, in other embodiments, the body 42 has at least one motor associated therewith, while the arms 46, 48 have no motors. In further alternative implementations, each of the body 42 and the arms 46, 48 has at least one motor associated therewith. In one embodiment, any of the motors disclosed or contemplated herein can be brush or brushless motors. Further, the motors can be, for example, 6 mm, 8 mm, or 10 mm diameter motors. Alternatively, any known size that can be integrated into a medical device can be used. In a further alternative, the actuators can be any known actuators used in medical devices to actuate movement or action of a component. Examples of motors that could be used for the motors described herein include the EC 10 BLDC+GP10A Planetary Gearhead, EC 8 BLDC+GP8A Planetary Gearhead, or EC 6 BLDC+GP6A Planetary Gearhead, all of which are commercially available from Maxon Motors, located in Fall River, MA. There are many ways to actuate these motions, such as with DC motors, AC motors, permanent magnet DC motors, brushless motors, pneumatics, cables to remote motors, hydraulics, and the like. As such, the actuation source can be at least one motor, hydraulic pressure source, pneumatic pressure source, or any other actuation source disposed remotely from or proximally to the device 40 such that an appropriate coupling or transmission mechanism (such as at least one cable, at least one hydraulic transmission hose, at least one pneumatic transmission hose, or any other transmission mechanism) is disposed through the body 42.

In one embodiment, the various joints discussed above in accordance with any of the embodiments disclosed or contemplated herein can be driven by electrical motors disposed within the device and, in some implementations, near each joint. Other embodiments include the incorporation of pneumatic or hydraulic actuators in any of the device implementations herein. In additional alternative embodiments, the driving actuators are disposed outside the device and/or body cavity and power transmission mechanisms are provided to transmit the energy from the external source to the various joints of any device herein. Such a transmission mechanism could, for example, take the form of gears, drive shafts, cables, pulleys, or other known mechanisms, or any combination thereof.

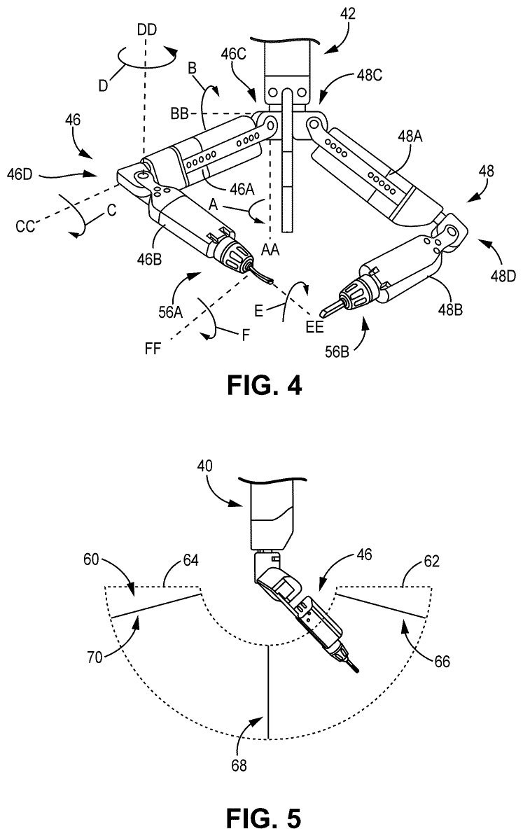

As discussed above and as shown in FIG. 4, the upper arms 46A, 48A are coupled to the body 42 via shoulder joints 46C, 48C. In one embodiment, each shoulder joint 46C, 48C is a joint made up of shoulder bodies 54A, 54B having two axes of rotation. For example, the right shoulder joint 46C can be configured to result in rotation of the upper arm 46A in relation to body 42 as shown by arrow A around axis AA (that is substantially parallel to the longitudinal axis of the body 42) and also as shown by arrow B around axis BB, which is substantially perpendicular to axis AA. Because left shoulder joint 48C and left upper arm 48A are substantially the same as the right shoulder joint 46C and the right upper arm 46A, the above description also applies to those substantially similar (or identical) components. Alternatively, any known joint can be used to couple the upper arms 46A, 48A to the body 42.

Continuing with FIG. 4, the upper arms 46A, 48A, according to one implementation, are coupled to the forearms 46B, 48B, respectively, at the elbow joints 46D, 48D such that each of the forearms 46B, 48B can rotate. For example, the right forearm 46B can rotate in relation to the upper arm 46A as shown by arrow C around axis CC (which is substantially parallel to the longitudinal axis of the upper arm 46A) and also can rotate in relation to the upper arm 46A as shown by arrow D around axis DD, which is substantially perpendicular to axis CC. Because left elbow joint 48D and left forearm 48B are substantially the same as the right elbow joint 46D and the right forearm 46B, the above description also applies to those substantially similar (or identical) components. Alternatively, any known joint can be used to couple the forearms 46B, 48B to the upper arms 46A, 48A.

Further, the right end effector 56A can also rotate relative to the forearm 46B as shown by arrow E around axis EE (which is substantially parallel to the longitudinal axis of the forearm 46B) such that the end effector 56A can “roll” in relation to the forearm 46B. Further, in those embodiments in which the end effector 56A has rotating components (such as jaws of a grasper, for example), the end effector 56A jaws can be actuated to move between at least two configurations, such as an open configuration and a closed configuration, as shown by arrow F around axis FF (which is substantially perpendicular to axis EE). Because left end effector 56B and left forearm 48B can be substantially the same as the right end effector 56A and right forearm 46B, the above description also applies to those substantially similar (or identical) components. Alternatively, the end effectors 56A, 56B can be coupled to the forearms 46B, 48B, respectively, such that the end effectors 56A, 56B can be moved or actuated in any known fashion.

In one embodiment, the upper arms 46A, 48A have a length of about 80 to 100 mm. Alternatively, the upper arms 46A, 48A have a length of about 90 mm. In certain implementations, the forearms 46B, 48B have a length of about 60 to about 80 mm. Alternatively, the forearms 46B, 48B have a length of about 70 mm. According to some embodiments, the end effectors 56A, 56B can add a length of about 15 to about 25 mm, or alternatively about 20 mm, of length to the end of the forearms 46B, 48B. Further, each of the arms 46, 48 have a width ranging from about 13 to about 15 mm, or alternatively a width of about 14 mm. In addition, each of the arms 46, 48 have a depth of about 20 mm to about 26 mm, or alternatively a depth of about 23 mm.

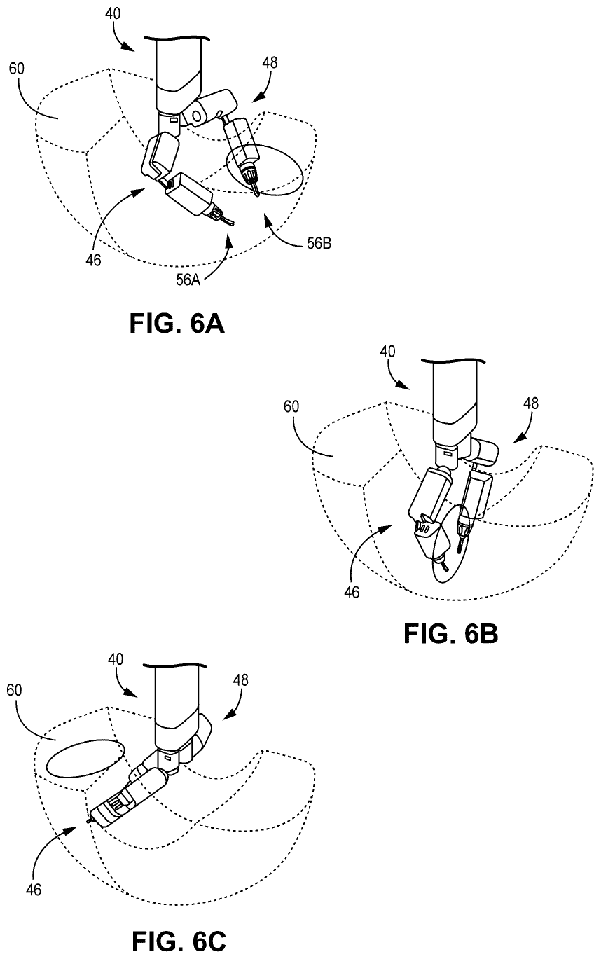

FIGS. 5-6C schematically depict the entire workspace 60 of the arms 46, 48 of a robotic device 40, according to certain embodiments. In these embodiments, “workspace” 60 means the space 60 around the robotic device 40 in which either arm 46, 48 and/or end effector 56A, 56B can move, access, and perform its function within that space.

More specifically, FIG. 5 depicts a side view of the device 40 and the entire workspace 60, including the full range of motion of the arms 46, 48 from its forward point 62 of +90° in the workspace 60 to its rearward point 64 of −90°. Further, FIG. 5 also shows the forward sweep angle of +75° (66), the neutral angle of 0° (68), and the rearward sweep angle of −75° (70). Similarly, FIG. 6A depicts the arms 46, 48 at the forward sweep angle, FIG. 6B depicts the arms 46, 48 at the neutral position, and FIG. 6C depicts the arms at the rearward sweep angle. Thus, each arm 46, 48 has a range of motion and corresponding workspace 60 that extends from the front of the device 40 to the back of the device 40. Thus, both arms 46, 48 move equally to the front and the back, through about 180° of space relative to the axis of the device body 42. This workspace 60 allows the robotic device 40 to work to the front and back equally well without having to reposition the body 42.

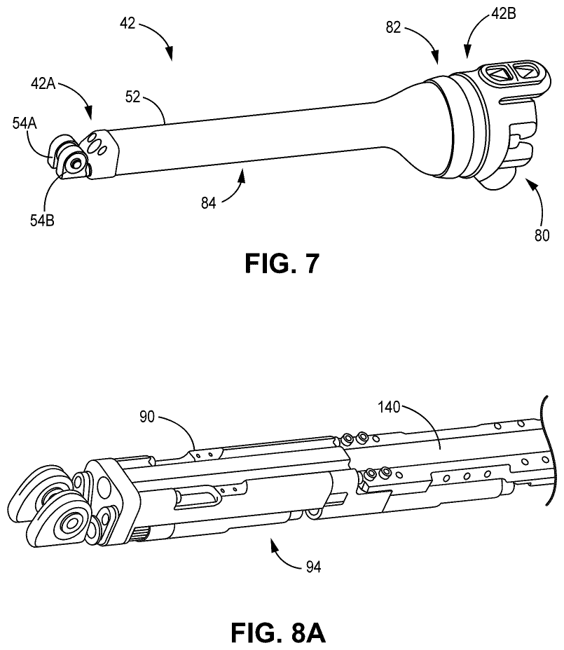

One embodiment of the device body 42 with the external casing or housing 52 is depicted in FIG. 7. The body 42 has a proximal camera port or nest 80 at its proximal end 42B, the right and left shoulder housings 54A, 54B rotatably coupled at its distal end 42A, and a groove 82 defined in an outer circumference of the housing 52 distal of the camera port 80 as shown. Further, the body 42 has a reduced diameter section or length 84 extending distally from a location distal of the groove 82 to the distal end 42A of the body 42. The reduced diameter section 84 has a width ranging from about 28 mm to about 30 mm. Alternatively, the reduced diameter section 84 has a width of about 29 mm. Further, the reduced diameter section 84 has a depth ranging from about 22 mm to about 24 mm. Alternatively, the reduced diameter section 84 has a depth of about 23 mm. In other words, the reduced diameter section can have a cross-sectional circumference ranging from about 100 mm to about 108 mm. In contrast, the proximal end 42B of the body 42 has a diameter ranging from about 50 mm to about 70 mm, or alternatively has a diameter of about 60 mm. Further, the reduced diameter length 84 has a length ranging from about 150 mm to about 250 mm. Alternatively, the reduced diameter section 84 has a length of about 200 mm. As mentioned above, in this specific implementation, it is the drivetrain configuration (as will be described in detail below) within the housing 52 that makes it possible for the radial diameter of the reduced diameter length 84 to be less than the radial diameter of the proximal end 42B, and, in certain implementations, to be at least 20 mm less than the diameter of the proximal end 42B, and in some cases, at least 30 mm or even 37 mm less.

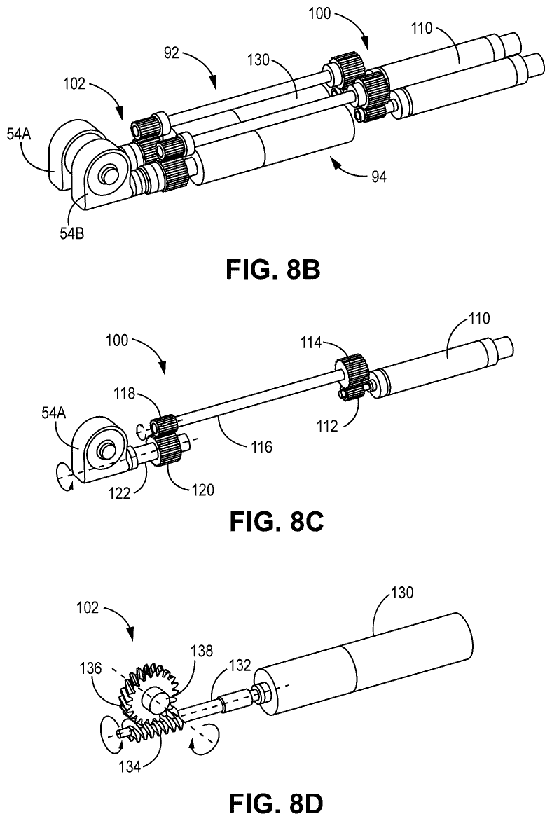

FIGS. 8A-8D, according to one embodiment, depict the internal components of the body 42, which is shown in these figures without its casing 52. More specifically, FIG. 8A depicts an internal support structure 90 such that the internal drivetrains 92, 94 and the camera lumen 140 as best shown in FIG. 8B are disposed within or associated with the internal structure 90. In contrast, FIGS. 8B-8D depict the internal actuation and control components of the body 42 with the housing or support components (such as the external housing 52 and the internal support structure 90) not shown in order to better display the drivetrains 92, 94. Each of these drivetrains 92, 94 are configured to provide two degrees of freedom at the shoulders 54A, 54B.

FIG. 8B depicts the right and left drivetrains 92, 94 that are coupled to the right and left shoulder housings 54A, 54B, respectively. Further, FIGS. 8C and 8D depict the separate pitch and roll drivetrains 100, 102 of the right drivetrain 92. It is understood that the components of the left drivetrain 94 that operate/control/actuate the left shoulder housing 54B are substantially the same as those depicted and described herein with respect to the right drivetrain 92 and right shoulder housing 54A and that the descriptions provided below apply equally to those components as well.

In one embodiment, as best shown in FIGS. 8C and 8D, the right shoulder drivetrain 92 is made up of two separate drivetrains: the shoulder roll drivetrain 100 and the shoulder pitch drivetrain 102. As shown in FIG. 8C, the shoulder roll drivetrain 100 is made up of the first or roll actuator 110, a first or roll motor gear 112 operably coupled to the actuator 110, a first driven gear 114 rotatably coupled to the roll motor gear 112, a driveshaft 116 fixedly attached to the first driven gear at a proximal end, and second driven gear 118 fixedly attached to the driveshaft 116 at a distal end of the driveshaft 116, and a shoulder gear 120 rotatably coupled to the second driven gear 118, wherein the shoulder gear 120 is fixedly attached (rotationally constrained) to the right shoulder 54A via a shoulder driveshaft 122.

In operation, actuation of the actuator 110 causes rotation of the driveshaft 116 (via the motor gear 112 and the first driven gear 114), which causes roll rotation of the right shoulder 54A (via the second driven gear 118, the shoulder gear 120, and the shoulder driveshaft 122). Thus, the roll actuator 110 causes roll rotation of the shoulder 54A around an axis that is substantially parallel to a longitudinal axis of the device body 42.

Further, as shown in FIG. 8D, the shoulder pitch drivetrain 102 of the right shoulder drivetrain 92 is made up of the second or pitch actuator 130, an elongate motor shaft 132 operably coupled to the actuator 130 and disposed through a lumen (not shown) in the shoulder gear 120 and shoulder driveshaft 122, a drive gear 134 attached (rotationally constrained) to the motor shaft 132, and a driven gear 136 rotatably coupled to the drive gear 134. In one embodiment, the drive gear 134 is a worm gear 134 and the driven gear 136 is a worm wheel 136. Alternatively, any combination of known gears can be used.

In operation, actuation of the actuator 130 causes rotation of the motor shaft 132, which causes pitch rotation of the shoulder 54A via the drive gear 134 and the driven gear 136. Thus, the pitch actuator 130 causes pitch rotation of the shoulder 54A around an axis that is substantially perpendicular to the axis of the roll rotation.

Because the left drivetrain 94 is substantially the same as the right drivetrain 92, the above description also applies to those substantially similar (or identical) components. Alternatively, any known drivetrain can be used to actuate the two axes of rotation of the shoulders 54A, 54B.

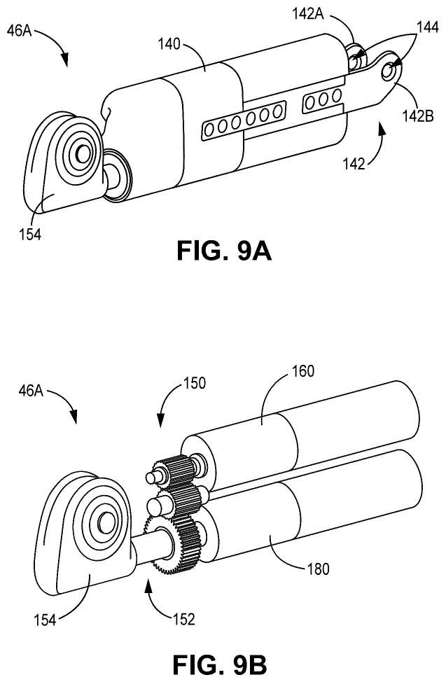

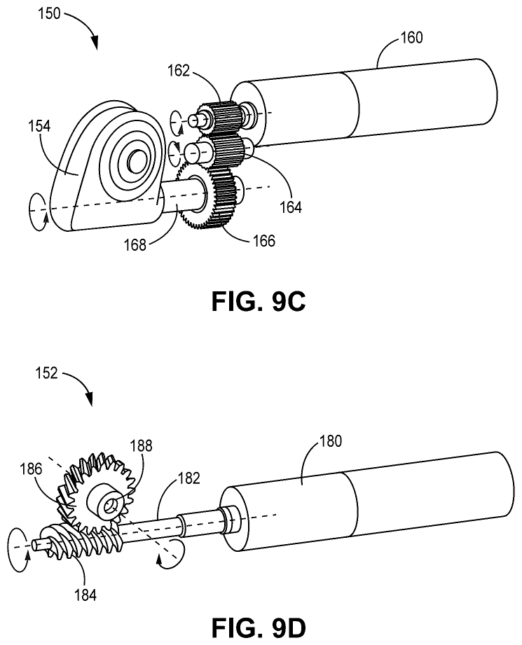

FIGS. 9A-9D, according to one embodiment, depict the right upper arm 46A in further detail. More specifically, FIG. 9A depicts the right upper arm 46A with its external housing 140 and proximal attachment structure 142, while FIGS. 9B-9D depict the upper arm 46A without its housing such that the internal actuators and drivetrains are visible. It is understood that the components of the left upper arm 48A are substantially the same as those depicted and described herein with respect to the right upper arm 46A and that the descriptions provided below apply equally to those components as well.

As shown in FIG. 9A, the proximal attachment structure 142 is made up of two attachment arms 142A, 142B extending from the proximal end of the housing 140. Each of the two arms 142A, 142B has a shoulder attachment opening 144 defined therein, such that the openings 144 receive the rotatable shaft of the driven gear 136 discussed above. Alternatively, the attachment structure 142 can be any known attachment mechanism or device for coupling the upper arm 46A to the shoulder 54A.

FIG. 9B depicts the separate roll 150 and rotation 152 drivetrains that are coupled to the right elbow housing 154. Further, FIG. 9C depicts the roll drivetrain 150 and FIG. 9D depicts the elbow rotation drivetrain 152. It is understood that the components and drivetrains of the left upper arm 48A are substantially the same as those depicted and described herein and that the descriptions provided below apply equally to those components as well.

In one embodiment, as best shown in FIGS. 9B and 9C, the roll drivetrain 150 is made up of the first or roll actuator 160, a first or roll motor gear 162 operably coupled to the actuator 160, a first driven gear 164 rotatably coupled to the roll motor gear 162, an elbow gear 166 rotatably coupled to the first driven gear 164, an elbow driveshaft 168 fixedly attached to the elbow gear 166, wherein the elbow gear 166 is fixedly attached (rotationally constrained) to the elbow housing (or “elbow assembly”) 154 via the elbow driveshaft 168. In one embodiment, the first drive gear 164 is a single gear 164 as shown. Alternatively, the first drive gear 164 can be made up of two gears rotationally constrained to each other (not shown).

In operation, actuation of the actuator 160 causes rotation of the driveshaft 168 (via the motor gear 162, the first driven gear 164, and the elbow gear 166), which causes roll rotation of the right elbow housing 154 (and the forearm 46B attached thereto). Thus, the roll actuator 160 causes roll rotation of the elbow housing 154 (and the forearm 46B) around an axis that is substantially parallel to a longitudinal axis of the upper arm 46A.

Further, in one implementation as best shown in FIGS. 9B and 9D, the elbow rotation drivetrain 152 is made up of the second or rotation actuator 180, an elongate motor shaft 182 operably coupled to the actuator 180 and disposed through a lumen (not shown) in the elbow gear 166 and elbow driveshaft 168, a drive gear 184 attached (rotationally constrained) to the motor shaft 182, and a driven gear 186 rotatably coupled to the drive gear 184. In one embodiment, the drive gear 184 is a worm gear 184 and the driven gear 186 is a worm wheel 186. Alternatively, any combination of known gears can be used.

In operation, actuation of the actuator 180 causes rotation of the motor shaft 182, which causes rotation of the forearm 46B via the drive gear 184 and the driven gear 186. Thus, the elbow rotation actuator 180 causes rotation of the elbow shaft 188 around an axis that is substantially perpendicular to the axis of the roll rotation.

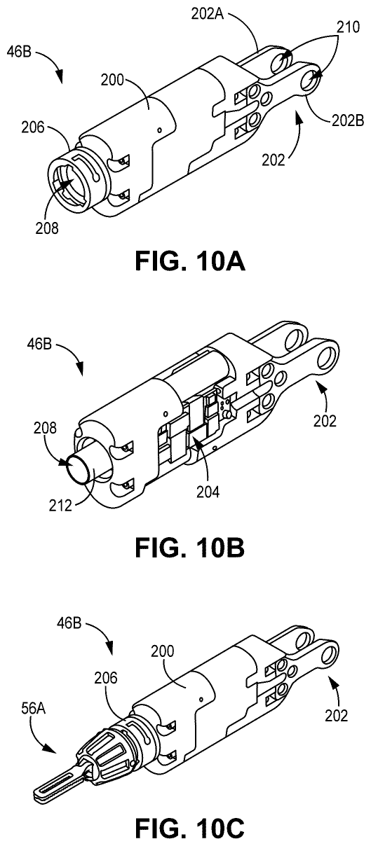

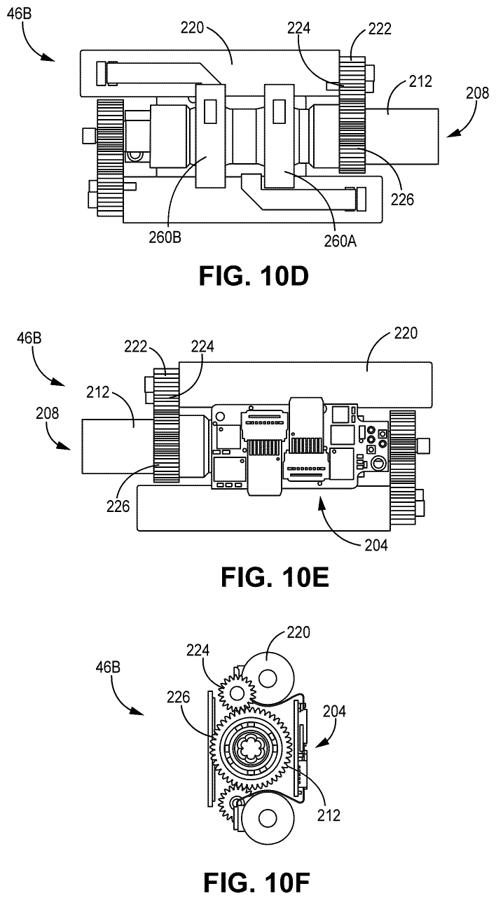

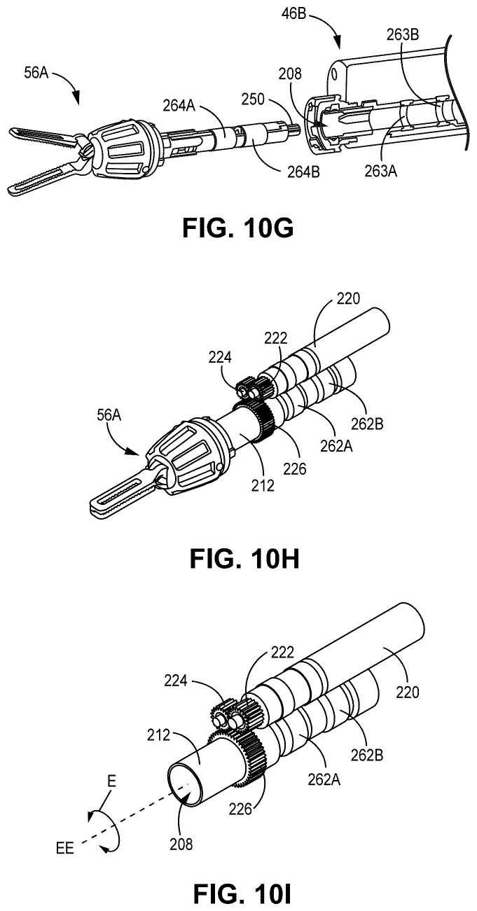

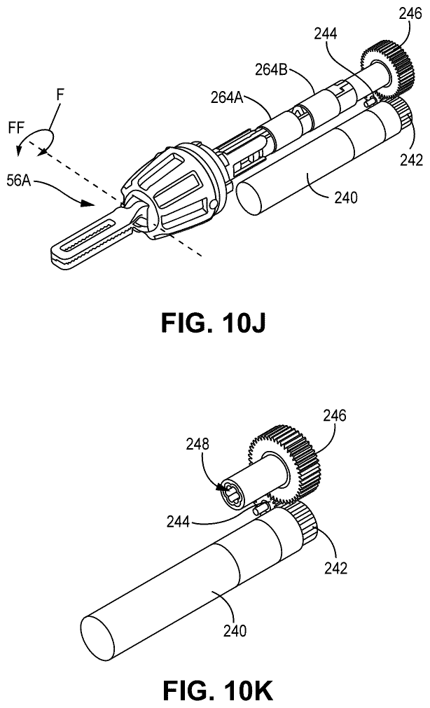

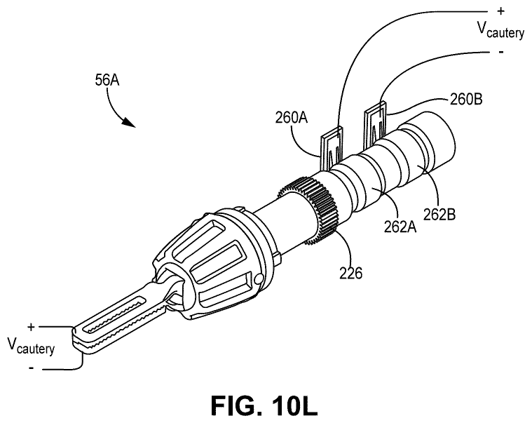

FIGS. 10A-10L depict various embodiments of the right forearm 46B and the right end effector 56A. FIGS. 10A-10C depict the right forearm 46B with most/all of its external housing 200, while FIGS. 10D-10F and 10H-10K show internal portions and/or components of the forearm 46B without its housing 200. The various implementations disclosed and depicted herein include the actuators, drive components, and electronics that can be used to accomplish both tool roll and tool drive (open/close action), as will be described in further detail below. As set forth below, the right forearm 46B also has at least one electrically isolated cautery circuit, enabling cautery end effectors. Certain embodiments are configured to allow for easy removal and replacement of an end effector (a “quick change” configuration), such as end effector 56A. Further embodiments contain sealing elements that help to prevent fluid ingress into the mechanism. It is understood that the components of the left forearm 48B and left end effector 56B are substantially the same as those depicted and described herein with respect to the right forearm 46B and right end effector 56A and that the descriptions provided below apply equally to those components as well.

As shown in FIG. 10A, according to one embodiment, the forearm 46B has a proximal attachment structure 202 for attachment to the elbow housing 154, an end effector coupling structure 206, and an end effector lumen 208 defined within a rotatable end effector tube (“roll tube”) 212. The end effector coupling structure 206 in this specific embodiment is a coupling collar 206 with female coupling features for receiving male features of the end effector 56A as discussed in detail below. The proximal attachment structure 202 is made up of two attachment arms 202A, 202B extending from the proximal end of the housing 200. Each of the two arms 202A, 202B has an elbow attachment opening 210 defined therein, such that the openings 210 receive the rotatable shaft 188 of the elbow housing 154 discussed above. Alternatively, the attachment structure 202 can be any known attachment mechanism or device for coupling the forearm 46B to the elbow 154.

In one implementation, as shown in FIG. 10B, a processor or controller 204 is disposed within the forearm housing 200 and is coupled to the motors and other components therein (as discussed in detail below) to control such motors and components. In one embodiment, the processor 204 is a printed circuit board. Alternatively, any known processor or controller 204 can be used.

FIG. 10C depicts the forearm 46B with the end effector 56A attached thereto.

FIGS. 10D and 10E depict opposite sides of the internal components of the forearm 46B, with FIG. 10D depicting the internal components of the right side and FIG. 10E depicting the internal components of the left side. FIG. 10F depicts an end view of those internal components. FIG. 10G depicts a cross-sectional view of the distal end of the forearm 46B with the end effector 56A positioned for insertion into or removal from the end effector lumen 208.

According to one implementation, certain of the internal components depicted in 10D-10F and 10H-10I are configured to actuate rotation of the end effector tube 210 (and the end effector 56A disposed therein) around axis EE (as best shown in FIGS. 4 and 10I), which is parallel to the longitudinal axis of the right forearm 46B. This rotation around axis EE is also referred to as “tool roll.”

The rotation, in one aspect, is created as follows. As best shown in FIGS. 10G-10I, a roll actuator 220 is provided that is, in this implementation, a motor assembly 220. The actuator 220 is operably coupled to the motor gear 222, which is a spur gear 222 in this embodiment. The motor gear 222 is coupled to the driven gear 224 such that rotation of the motor gear 222 causes rotation of the driven gear 224. The driven gear 224 is rotatably coupled to the tube gear 226, which is fixedly coupled to the roll tube (or hub) 212. The lumen 208 of the roll tube 212 has end effector coupling features 230 defined in the inner wall of the lumen 208 (as best shown in FIG. 10G) such that the tube 212 is rotationally constrained to the end effector 56A when the end effector 56A is positioned within the lumen 208.

In operation, actuation of the roll actuator 220 causes rotation of the roll tube 212 via the motor gear 222, the driven gear 224, and the tube gear 226, which causes rotation of the end effector 56A around axis EE (as best shown in FIG. 10I).

According to one implementation, certain of the internal components depicted in 10D-10F and 10J-10K are configured to actuate the end effector 56A to open and close around axis FF (as best shown in FIGS. 4 and 10J), which is transverse to the longitudinal axis of the right forearm 46B. This rotation around axis FF is also referred to as “tool drive.”

The rotation, in one aspect, is created as follows. As best shown in FIG. 10J and 10K, a tool actuator 240 is provided that is, in this implementation, a motor assembly 240. The actuator 240 is operably coupled to the motor gear 242, which is a spur gear 242 in this embodiment. The motor gear 242 is coupled to the driven gear 244 such that rotation of the motor gear 242 causes rotation of the driven gear 244. The driven gear 244 is rotatably coupled to the interface gear 246 such that rotation of the driven gear 244 causes rotation of the interface gear 246. The interface gear 246 is fixedly coupled to a female drive interface 248, which in one embodiment is a Torx interface 248. The female drive interface 248 is mateable with a male drive interface 250 disposed at a proximal end of the end effector 56A (as best shown in FIG. 10G) such that the female drive interface 248 is rotationally constrained to the male drive interface 250 when the end effector 56A is positioned within the lumen 208.

In operation, actuation of the tool actuator 240 causes rotation of the female drive interface 246 via the motor gear 242, the driven gear 244, and the interface gear 246, which causes rotation of the male drive interface 248, which is operably coupled to the graspers of the end effector 56A. Thus, depending on the specific end effector 56A and the configuration thereof, rotation of the female drive interface 248 can cause rotation of the male drive interface 250, which can cause rotation of the graspers around axis FF.

As noted above, the forearm 46B (and, in certain embodiments, forearm 48B) also has at least one electrically isolated cautery circuit, thereby enabling cautery end effectors. As best shown in FIG. 10L, according to one embodiment, electrical contacts 260A, 260B are provided within the forearm 46B that are positioned such that the external contact rings 262A, 262B disposed around the roll tube 212 remain in contact with the corresponding contacts 260A, 260B during rotation of the tube 212. More specifically, first electrical contact 260A is disposed within the forearm 46B such that it is adjacent to and in contact with the first external contact ring 262A, while second electrical contact 260B is disposed such that it is adjacent to and in contact with the second external contact ring 262B. Thus, the rings 262A, 262B are in contact with and can receive electrical current from the contacts 260A, 260B, respectively, while the roll tube 212 is rotating or stationary.

Further, as best shown in FIG. 10G, each of the external contact rings 262A, 262B have an inner contact surface 263A, 263B that is disposed within the lumen 208 of the roll tube 212. That is, each ring 262A, 262B extends radially through the roll tube such that the inner contact surface 263A, 263B is disposed along and flush with the inner wall of the roll tube 212. As such, the inner contact surfaces 263A, 263B are disposed within the lumen 208 such that surfaces 263A, 263B remain in contact with the corresponding tool contacts 264A, 264B disposed on the outer surface of the end effector 56A when the end effector is coupled to the forearm 46B. More specifically, when the end effector 56A is coupled to the forearm 46B, first inner contact surface 263A is disposed at a point along the length of the lumen 208 such that it is adjacent to and in contact with the first tool contact 264A while second inner contact surface 263B is disposed at a point along the length of the lumen 208 such that it is adjacent to and in contact with the second tool contact 264B.

In operation, when electrical current is required for a cauterization end effector (such as end effector 56A, for example), electrical current can be applied to one or both of the electrical contacts 260A, 260B as needed. This current will be transferred from one or both of the contacts 260A, 260B to one or both of the external contact rings 262A, 262B electrically coupled thereto. As a result, the electrical current will pass through one or both of the inner contact surfaces 263A, 263B, and thus, if an end effector such as end effector 56A is coupled to the forearm 46B, the electrical current will then transfer to one or both of the tool contacts 264A, 264B on the forearm 46B and thus can be used to perform cauterization as needed with the cauterization end effector (such as end effector 56A).

One of the unique aspects of this device configuration that makes it possible to reduce the overall axial diameter of both the device body 42 and the arms 46, 48 is the two degree of freedom drivetrain modules that are used in both the body 42 and the upper arms 46A, 48A. For purposes of this discussion, the drivetrain modules of the right upper arm 46A will be used, but it is understood that the same general drivetrain configuration is used in the device body 42 as well. That is, the general configuration of the two drivetrains (as shown, for example, in FIGS. 8B-8D and 9B-9D) includes a first drivetrain (such as drivetrain 100 and drivetrain 150) that drives a roll joint and a second drivetrain (such as drivetrain 102 and drivetrain 152 that drives a pitch movement. In both the body 42 and the upper arms 46A, 48A, the two drivetrains are strategically positioned in relation to each other such that the radial diameter of the body/arm is minimized, thereby minimized the size of the incision or port required to position the device into the patient.

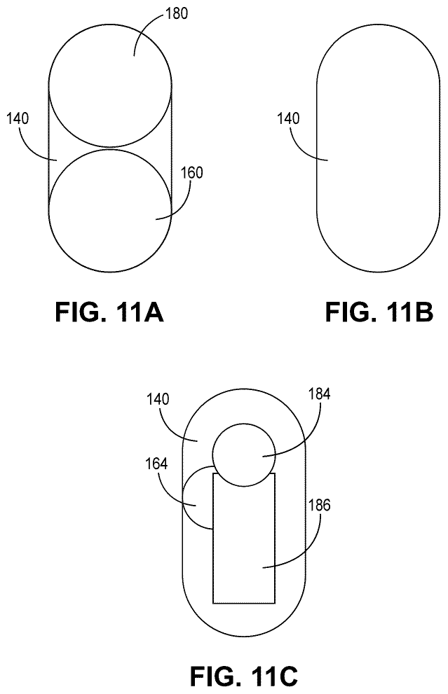

Given that the motors (actuators) in the device embodiments herein are cylindrical and thus have a circular radial cross-section, the placement of these motors adjacent to one another as shown in FIG. 11A is the tightest packing of two circles to minimize the radial profile. Using the specific example of the right upper arm 46A, the two circles in FIG. 11A represent the pitch drive motor 180 and the roll drive motor 160 as discussed above, and thus the oval shape 140 that contains both motors 160, 180 with the least amount of space represents the upper arm housing 140. Similarly, the motors 110, 130 of the drivetrain 92 in the device body 42 as discussed above are positioned in a similar fashion (but with the roll motor 110 positioned proximally of the pitch motor 130).

Further, as shown in FIG. 11C, the arrangement of the gears in relation to the motors 160, 180 results in the gears being disposed within the radial cross section of the motors 160, 180 (when the device is in the straight insertion configuration). More specifically, the driven gear(s) 164 of the roll drivetrain and the motor gear 184 and the driven gear 186 of the pitch drivetrain, along with any other gears of the drivetrains, are disposed within the upper arm housing 140.

Thus, as discussed in detail above, the configuration of the motors 160, 180 and the related gears within the upper arm 46A results in the roll motor 160 causing rotation of the shoulder 54A around a roll axis that is parallel to the axis of the motor 160 (and motor 180), and further results in the pitch motor 180 causing rotation of the shoulder rotatable shaft 138 around an axis that is substantially perpendicular to the axis of the motor 180 (and motor 160).

This basic drivetrain configuration is used in both the body 42 and the upper arm 46A. With respect to the configuration in the body 42, the additional drive shaft 116 is used in the roll drivetrain to maintain the same cross section (or further reduce the cross section) at the expense of length. This allows for larger motors to be used in the device body 42 while maintaining the same cross section.

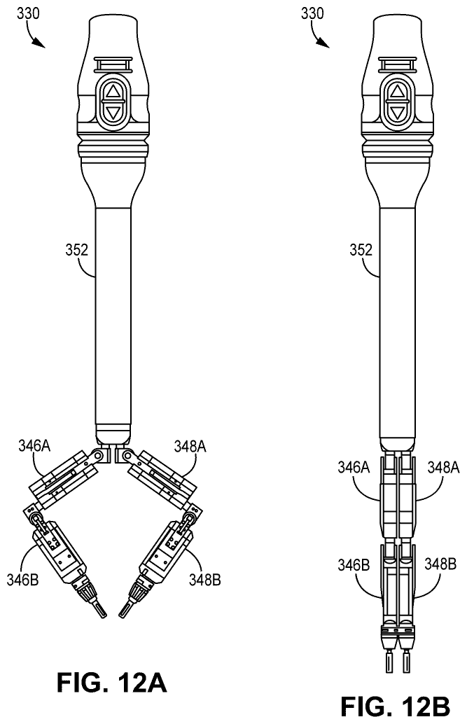

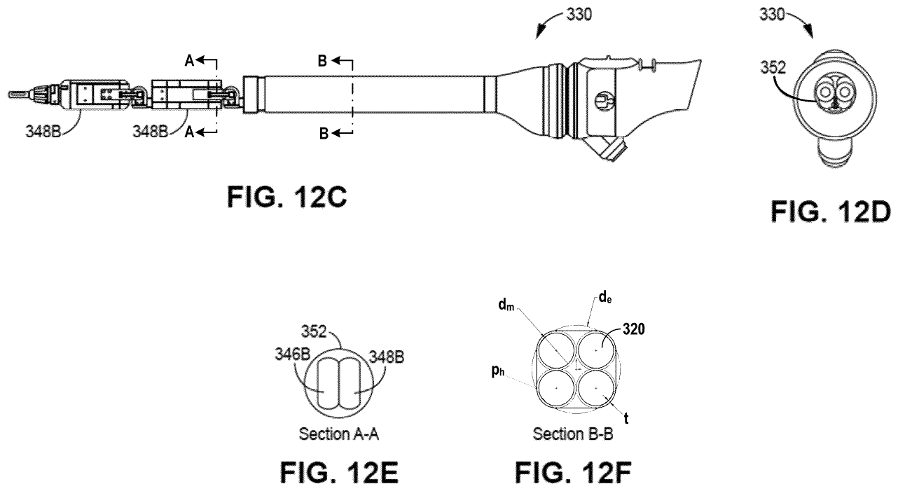

In some embodiments and shown in FIGS. 12A-12F, the configuration of the device can be designed to have a smaller insertion diameter. In such embodiments, the topology and kinematics are identical to those described above. This can be accomplished by using smaller motor actuators (e.g., 6 mm diameter motors instead of 8 mm diameter motors) the use of bushings which have a smaller radial dimension than ball bearings, changes in gearing, and/or with changes in cable routing and printed circuit boards. The device is shown in FIG. 12A in a working configuration and in FIGS. 12B-F an elongated configuration for insertion and extraction in and out of the abdominal cavity. In the insertion and extraction configuration the device arms are configured within the cross section of the base link.

In such embodiments as shown in FIGS. 12B, the width of arms 346A-B and 348A-B is narrower than the width of the body 352. As shown in FIG. 12C, the height of the arms 348A-B (and 346A-B although hidden in the Figure) is less than the height of body 352. In such embodiments, when the robotic device 330 is in the insertion configuration the arms 346A-B and 348A-B fit within the exterior perimeter of the body 352 during the insertion process.

This is depicted in detail in the cross sections of FIGS. 12E and 12F through two areas of the robotic device 330 and detail the perimeters of the arms 346B, 348B as compared to the exterior perimeter of the body 352. In some embodiments, the body 352 is a cylinder (for at least the position of the body to be inserted through the port) having a substantially circular diameter of about 15-30 mm. The circular perimeter of the body 352 is much easier to seal with the port and introduces less stress on the incision for the patient.

Looking closer at FIG. 12F, depicts a schematic view of an embodiment of motor pairs 320 within upper arms 346A, 348A. In the embodiment shown, each upper arm 346A, 348A includes two motors 320, combining to form a matrix of four motors 320. Each of the motors 320 in the matrix of motors has a motor diameter dₘ and a thickness t. The motor diameters dₘ and motor thicknesses t combine to define an effective diameter of the robotic device dₑ and hull perimeter pₕ. The effective diameter dₑ and the hull perimeter pₕ define the size of the port that the robotic device can fit through. The wall thickness t allows for a 1 mm distance between the two arms and may include structure to hold the motors, cabling, protective covers, and/or other items.

While other motor diameters may be used beside those shown, Table 1 below defines these dimensions based on certain chosen motor diameters.

TABLE 1

| | | | |
|---|---|---|---|
| | Assumed wall/ | | |
| Motor | structure | Perimeter of | Effective |
| Diameter | thickness | convex hull | diameter |

| | | | | |
|---|---|---|---|---|
| 4 | mm | 0.5 mm | 34.71 mm | 11.05 mm |
| 6 | mm | 0.5 mm | 48.99 mm | 15.59 mm |
| 8 | mm | 0.5 mm | 63.27 mm | 20.14 mm |
| 10 | mm | 0.5 mm | 77.56 mm | 24.69 mm |
| 12 | mm | 0.5 mm | 91.84 mm | 29.23 mm |
| 13 | mm | 0.5 mm | 98.98 mm | 31.51 mm |

A person of ordinary skill in the art would readily recognize that in addition to other non-standard motor sizes, motors with different sizes may be combined in any combination without deviating from the scope of the disclosure. Further, other combinations of arms (e.g., single arm, three arms, etc.) may be combined with any combination of motor sizes to achieve a desired result without deviating from the scope of the disclosure.

In accordance with certain implementations, another benefit of the device configurations herein is that the shoulder housings (such as right shoulder housing 54A) and the elbow housings (such as right elbow housing 154) and the joints created by those housings are either radially symmetric or mirror images. Further, the left and right sides (arms and drivetrains) operate independently of one another. As a result, the arms 46, 48 are essentially modular, meaning that various device embodiments as contemplated herein can have one arm, two arms, three arms, four arms, or any additional number of arms.

For example, a one-arm robotic device 280 embodiment is depicted in FIG. 12. The device 280 can be used independently or in cooperation with other surgical devices. The arm 282 of the device 280 operates in a fashion similar to the individual arms as described herein (such as the right arm 36, as discussed in detail above). As such, the arm 282 can be positioned in a straight configuration (such as for insertion or extraction) or any other configuration that is possible based on the components described above.

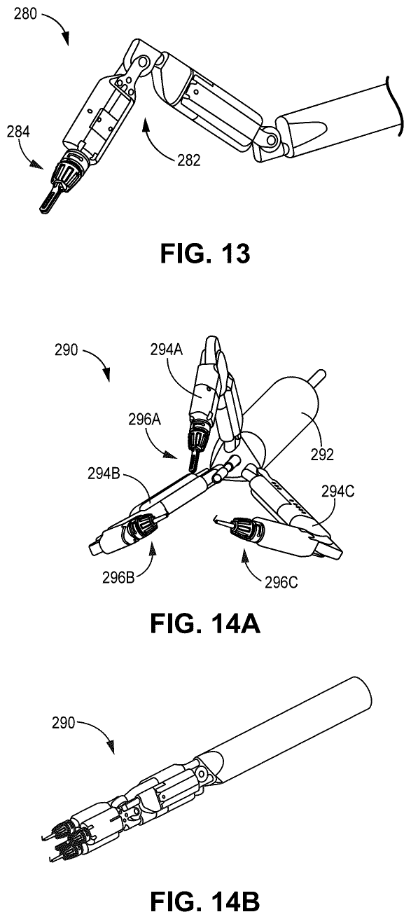

In another example, a three-arm robotic device 290 is shown in FIGS. 13A and 13B, according to a further implementation. The device 290 can be used independently or in cooperation with other surgical devices. According to one embodiment, the device 290 can have three arms 294A, 294B, 294C permanently attached to the device body 292 as shown. Alternatively, the arms 294A, 294B, 294C are modular, such that they can be easily and quickly attached to or detached from the body 292 in various numbers and configurations. In addition, according to certain embodiments, the elongate body 292 has one or more moveable sections where one or more of the arms 294A-C are attached such that one or more of the arms 294A-C are moveable in relation to the other arms such that each arm can be positioned deeper or shallower in relation to the other arms.

Further, while grasper end effectors 296A, 296B, 296C are shown, it is understood that any end effectors can be used with this device 290.

The arms 294A-C of the device 290 operate in a fashion similar to the individual arms as described herein (such as the right arm 36, as discussed in detail above). As such, the arms 294A-C can be positioned in a straight configuration (such as for insertion or extraction) as shown in FIG. 13B or any other configuration that is possible based on the components described above.

While the various systems described above are separate implementations, any of the individual components, mechanisms, or devices, and related features and functionality, within the various system embodiments described in detail above can be incorporated into any of the other system embodiments herein.

The term “about,” as used herein, refers to variation in the numerical quantity that can occur, for example, through typical measuring techniques and equipment, with respect to any quantifiable variable, including, but not limited to, mass, volume, time, distance, wave length, frequency, voltage, current, and electromagnetic field. Further, there is certain inadvertent error and variation in the real world that is likely through differences in the manufacture, source, or precision of the components used to make the various components or carry out the methods and the like. The term “about” also encompasses these variations. The term “about” can include any variation of 5% or 10%, or any amount—including any integer—between 0% and 10%. Further, whether or not modified by the term “about,” the claims include equivalents to the quantities or amounts.

Numeric ranges recited within the specification are inclusive of the numbers defining the range and include each integer within the defined range. Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1%, and 4 ¾ This applies regardless of the breadth of the range.

Although the various embodiments have been described with reference to preferred implementations, persons skilled in the art will recognize that changes may be made in form and detail without departing from the spirit and scope thereof.

What is claimed is:

1. A robotic surgical device comprising
(a) an elongate device body, the body comprising:
(i) a cylindrical section having a distal section diameter ranging from about 25 mm to about 30 mm; and
(ii) a proximal section having a proximal section diameter that is greater than a distal section diameter;
(b) a first arm operably coupled to a distal end of the elongate device body via a first shoulder assembly; and
(c) a second arm operably coupled to the distal end of the elongate device body via a second shoulder assembly.
